(19) United States
(12) Patent Application Publication
(10) Pub. No.: US 2023/0165283 A1
(43) Pub. Date: Jun. 1, 2023

(54) NATURAL FLAVOR COMPLEX FROM STEVIA REBAUDIANA PLANTS

(71) Applicant: PURECIRCLE USA INC., Westchester, IL (US)

(72) Inventor: Siddhartha PURKAYASTHA, Chicago, IL (US)

(21) Appl. No.: 17/917,476

(22) PCT Filed: Apr. 16, 2021
(86) PCT No.: PCT/US2021/027761

§ 371 Date: Oct. 6, 2022

Related U.S. Application Data
(60) Provisional application No. 63/011,261, filed on Apr. 16, 2020.

(51) Int. Cl. A23L 2/60; A23L 27/30; A23L 33/105; A61K 36/28; A61K 31/216

(52) U.S. Cl. CPC A23L 2/60; A23L 27/36; A23L 33/105; A61K 36/28; A61K 31/216

(57) ABSTRACT

A natural flavor complex obtained from Stevia plants is described. The natural flavor complex functions to modify the flavors of a consumable product without imparting detectable sweetness to the product.

### FIELD OF THE INVENTION

This invention relates to food ingredients obtained from Stevia rebaudiana plants, and their use in food products, beverages, and other consumables.

### BACKGROUND

Stevia rebaudiana Bertoni is a perennial shrub of the Asteraceae (Compositae) family native to certain regions of South America. The leaves of the plant contain from 10 to 20% of diterpene glycosides, which are around 150 to 450 times sweeter than sugar. The leaves have been traditionally used for hundreds of years in Paraguay and Brazil to sweeten local teas and medicines.

At present, there are more than 230 Stevia species with significant sweetening properties. The plant has been successfully grown under a wide range of conditions from its native subtropics to the cold northern latitudes.

The extract of Stevia rebaudiana plant contains a mixture of different sweet diterpene glycosides, which have a single base—steviol—and differ by the presence of carbohydrate residues at positions C13 and C19. These glycosides accumulate in Stevia leaves and compose approximately 10%-20% of the total dry weight.

Steviol glycosides have zero calories and can be used wherever sugar is used. They are ideal for diabetic and low-calorie diets.

It is noted that in the process of manufacture steviol glycoside sweeteners, large amounts of other constituents of the Stevia plant are also extracted with water. These other constituents are mainly separated during downstream processing and discarded. It is therefore advantageous to isolate, identify and utilize other constituents from the Stevia plant to optimize operational efficiency.

In WIPO publication number WO2018102447A2, which is incorporated by reference in its entirety herein, a process for producing food ingredients from Stevia rebaudiana plants and their use in food and beverage products is discussed. Among the food ingredients described are chlorogenic acids and their derivatives that naturally occur in the plant. These include, but are not limited to: neo-chlorogenic acid (neo-CGA; 5-O-caffeoylquinic acid or 5-CQA), crypto-chlorogenic acid (crypto-CGA; 4-O-caffeoylquinic acid or 4-CQA), n-chlorogenic acid (n-CGA; 3-O-caffeoyl quinic acid or 3-CQA), iso-chlorogenic acid A (iso-CGA A; 3,5-dicaffeoylquinic acid) iso-chlorogenic acid B (iso-CGA B; 3,4-dicaffeoylquinic acid), iso-chlorogenic acid C (iso-CGA C; 4,5-dicaffeoylquinic acid), and combinations thereof. Other ingredients described include, but are not limited to, flavonoids and other phenolic compounds.

### SUMMARY

The present invention is directed to the use of extracts of Stevia rebaudiana leaves and other parts of the plant, such extracts comprising chlorogenic acids and steviol glycosides obtained or derived from the Stevia plant, collectively referred to as “natural flavor complex” or “NFC,” as a flavoring with modifying properties (“FMP”) for food, beverages, and other consumable applications.

It was unexpectedly discovered that Stevia-derived natural flavor complexes provide desirable flavor modification without imparting (e.g. adding) detectable sweetness to a consumable.

It is to be understood that both the foregoing general description and the following detailed description are exemplary and explanatory, and are intended to provide further explanation of the invention as claimed.

### DETAILED DESCRIPTION

Advantages of the present invention will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

Those of skill in the art will also recognize that one or more of the process steps described below may be omitted. Those experienced in the art will also understand that although the process described below assumes certain order of the described steps, this order can be altered in some cases.

The flavor modifying properties of Stevia-derived flavor complexes were evaluated using the protocol set forth by the Flavor and Extract Manufacturers' Association (FEMA) Science Committee's Sensory Guidance Document, November 2013, (“FEMA Guidance”). Test 1 as described in the Guidance Document “is used to demonstrate that the FMP does not have an inherent sweetness or saltiness under conditions of intended use as an FMP in the finished food product.” (FEMA Guidance at page 2).

It was unexpectedly discovered that the natural flavor complex obtained from the leaves of the Stevia rebaudiana Bertoni plant has FMP properties without imparting (e.g. adding) an inherent or detectable sweetness to the consumable product.

Composition of Stevia-Derived Natural Flavor Complex (NFC)

In an embodiment, the Stevia-derived NFC comprises at least one molecule obtained from a Stevia rebaudiana Bertoni plant, selected from phenolic compounds, polyphenols, flavonoids, quinic and caffeic acids and their derivatives, neo-chlorogenic acid (neo-CGA; 5-O-caffeoylquinic acid or 5-CQA), crypto-chlorogenic acid (crypto-CGA; 4-O-caffeoylquinic acid or 4-CQA), n-chlorogenic acid (n-CGA; 3-O-caffeoylquinic acid or 3-CQA), iso-chlorogenic acid A (iso-CGA A; 3,5-dicaffeoylquinic acid) iso-chlorogenic acid B (iso-CGA B; 3,4-dicaffeoylquinic acid), iso-chlorogenic acid C (iso-CGA C; 4,5-dicaffeoylquinic acid), other chlorogenic acids and iso-chlorogenic acids known to art, retinoids, pigments, polysaccharides, oligosaccharides, disaccharides, monosaccharides, volatile oil components, sterols, terpenoids, sesquiterpenoids, diterpenes, triterpenes, coumarins, fatty acids and their derivatives, amino acids and their derivatives, dipeptides, oligopeptides, polypeptides, proteins, austroinulin, quercetin, sterebins, spathulenol, decanoic acid, 8,11,14-ecosatrienoic acid, 2-methyloctadecane, pentacosane, octacosane, stigmasterol, bsitosterol, a- and b-amyrine, Iupeol, b-amyrin acetate, pentacyclic triterpene and/or glycosides thereof, steviol glycosides, and combinations thereof.

As used herein, the term “chlorogenic acid” or “CGA” shall mean any one or more of neo-chlorogenic acid (neo-CGA; 5-O-caffeoylquinic acid or 5-CQA), crypto-chlorogenic acid (crypto-CGA; 4-O-caffeoylquinic acid or 4-CQA), n-chlorogenic acid (n-CGA; 3-O-caffeoylquinic acid or 3-CQA), iso-chlorogenic acid A (iso-CGA A; 3,5-dicaffeoylquinic acid) iso-chlorogenic acid B (iso-CGA B; 3,4-dicaffeoylquinic acid), iso-chlorogenic acid C (iso-CGA C; 4,5-dicaffeoylquinic acid), other chlorogenic acids and iso-chlorogenic acids known to the art.

In one embodiment, the NFC comprises at least a chlorogenic acid and a steviol glycoside extracted from the Stevia rebaudiana Bertoni plant. Typically, on a dry weight basis, the four major glycosides found in the leaves of Stevia are Dulcoside A (0.3%), Rebaudioside C (0.6-1.0%), Rebaudioside A (3.8%) and Stevioside (9.1%). Other glycosides identified in Stevia extract include Rebaudiosides B, C, D, E, F, M, AM, N, and O, Steviolbioside, Rubusoside, Stevioside, and minor steviol glycosides. Table 3 identifies additional steviol glycosides.

In a particular embodiment, the chlorogenic acid is present in the natural flavor complex at a level of at least about 15% by weight, and total steviol glycosides are present at a level of about 20% or less by weight. In another embodiment, the chlorogenic acid is present in the natural flavor complex at a level ranging from about 15% to about 40% by weight, and total steviol glycosides are present in the natural flavor complex in an amount ranging from about 0% to about 20% by weight. In another embodiment, the chlorogenic acid is present in the natural flavor complex at a level ranging from about 15% to about 40% by weight, and total steviol glycosides are present in the natural flavor complex in an amount ranging from greater than 0% to about 20% by weight. In another embodiment, the chlorogenic acid is present in the natural flavor complex at a level ranging from about 10% to about 50% by weight, and total steviol glycosides are present in the natural flavor complex in an amount ranging from greater than 0% to about 25% by weight. In another embodiment, the chlorogenic acid is present in the natural flavor complex at a level ranging from about 1% to about 80% by weight, and total steviol glycosides are present in the natural flavor complex in an amount ranging from greater than 0% to about 25% by weight.

In certain embodiments, the natural flavor complex comprises at least a chlorogenic acid, a flavonoid compound, and a steviol glycoside, all extracted from the Stevia rebaudiana Bertoni plant.

In another embodiment, the stevia extract FMP comprises at least a chlorogenic acid, a steviol glycoside, and at least one molecule selected from Tricaffeoylquinic acids; Rutin; Feruloyl-caffeoylquinc acids; Caffeoyl-feruloylquinic acids; Quercetin-glycoside; Galuteolin; Quercitrin; Roseoside; Feruloylquinic acids; Coumaroylquinic acids; Caffeoylshikimic acids; Caffeoylshikimic acids; 5-Caffeoylshikimic acid; Catechin; Luteolin; Trans-ferulic acid; and combinations thereof.

In yet another embodiment, the stevia extract FMP comprises at least a chlorogenic acid, a steviol glycoside, and at least one molecule selected from flavonoids, retinoids, pigments, polysaccharides, oligosaccharides, disaccharides, monosaccharides, volatile oil components, sterols, terpenoids, sesquiterpenoids, diterpenes, triterpenes, coumarins, fatty acids and their derivatives, amino acids and their derivatives, dipeptides, oligopeptides, polypeptides, proteins, austroinulin, quercetin, sterebins, spathulenol, decanoic acid, 8,11,14-ecosatrienoic acid, 2-methyloctadecane, pentacosane, octacosane, stigmasterol, bsitosterol, a- and b-amyrine, Iupeol, b-amyrin acetate, pentacyclic triterpene and glycosides thereof, and combinations thereof.

The natural flavor complex was obtained by the methods described in WO2018102447A2. The NFC obtained can be used in any consumable product as a flavoring with modifying properties, without imparting (e.g. adding) detectable sweetness to the consumable product. The NFC can be added to the consumable product in an amount ranging from 25 ppm to 1500 ppm, or even higher levels as long as the level is at or below the sweetness detection threshold in the particular consumable product.

In an embodiment, the flavor of the consumable product is modified by reducing bitterness or astringency or sweet aftertaste or bitter aftertaste perception of the consumable product as compared to a control product that does not contain the stevia extract FMP.

In another embodiment, the flavor of the consumable product is modified by enhancing one or more flavor notes as compared to a control product that does not contain the stevia extract FMP.

In another embodiment, the flavor of the consumable product is modified by suppressing one or more flavor notes as compared to a control product that does not contain the stevia extract FMP.

Table 1 summarizes certain chlorogenic acid and steviol glycoside constituents of natural flavor complexes obtained Stevia rebaudiana Bertoni leaves.

TABLE 1

| | | | | | | Sample | Useful |
|---|---|---|---|---|---|---|---|
| | Sample 1 | Sample 2 | Sample 3 | Sample 4 | Sample 5 | Mean | Ranges |
| Constituent | Weight % | Weight % | Weight % | Weight % | Weight % | Weight % | Weight % |
| neo-CGA | 2.07 | 0.63 | 1.22 | 1.98 | 0.60 | 1.30 | 0.5-3.0 |
| n-CGA | 9.12 | 1.92 | 9.07 | 8.01 | 9.16 | 7.46 | 1.5-10.0 |
| crypto-CGA | 2.10 | 1.47 | 1.98 | 2.55 | 1.66 | 1.95 | 1.0-4.0 |
| iso-CGA B | 1.13 | 4.48 | 2.04 | 2.32 | 1.35 | 2.26 | 0.5-6.0 |
| iso-CGA A | 10.26 | 4.03 | 13.54 | 5.48 | 14.54 | 9.57 | 3.5-16.0 |
| iso-CGA C | 3.04 | 7.08 | 7.58 | 4.93 | 6.33 | 5.79 | 1.5-10.0 |
| Total CGA | 27.72 | 19.61 | 35.43 | 25.27 | 33.64 | 28.33 | 15.0-40.0 |
| (inclusive of | | | | | | | |
| the above) | | | | | | | |
| Steviol | 4.12 | 15.44 | 5.45 | 1.53 | 9.76 | 7.26 | 0.5-17.0 |
| glycosides | | | | | | | |
| TOTAL | 31.84 | 35.05 | 40.88 | 26.80 | 43.40 | 35.59 | |

Table 2 summarizes certain chlorogenic acid and steviol glycoside constituents of natural flavor complexes obtained from Stevia rebaudiana Bertoni leaves. “TCGA” refers to total chlorogenic acid content, and “TSG” refers to total steviol glycoside content.

TABLE 2

| | neo- | n- | crypto- | iso- | iso- | iso- | | |
|---|---|---|---|---|---|---|---|---|
| Sample | CGA | CGA | CGA | CGA B | CGA A | CGA C | TCGA | TSG |
| Number | wt/wt | wt/wt | wt/wt | wt/wt | wt/wt | wt/wt | wt/wt | wt/wt |
| Sample 6 | 0.86 | 3.22 | 1.09 | 3.13 | 5.89 | 8.52 | 22.71 | 15.37 |
| Sample 7 | 0.63 | 1.92 | 1.47 | 4.48 | 4.03 | 7.08 | 19.61 | 15.44 |
| Sample 8 | 0.6 | 9.16 | 1.66 | 1.35 | 14.54 | 6.33 | 33.64 | 9.76 |
| Sample 9 | 1.22 | 9.07 | 1.98 | 2.04 | 13.54 | 7.58 | 35.43 | 5.45 |
| Sample 10 | 2.71 | 7.75 | 3.52 | 0.67 | 3.99 | 1.56 | 20.2 | 0.91 |
| Sample 11 | 2.54 | 8.91 | 2.24 | 0.87 | 6.67 | 1.78 | 23.01 | 2.8 |
| Sample 12 | 2.07 | 9.12 | 2.1 | 1.13 | 10.26 | 3.04 | 27.72 | 4.12 |
| Sample 13 | 1.61 | 8.79 | 2.95 | 5.01 | 7.62 | 9.23 | 35.21 | 2.53 |
| Sample 14 | 2.12 | 9.33 | 2.86 | 2.86 | 7.04 | 1.77 | 25.98 | 0.84 |
| Sample 15 | 2.03 | 7.5 | 2.43 | 1.63 | 6.39 | 3.88 | 23.86 | 6.17 |
| Sample 16 | 1.98 | 8.01 | 2.55 | 2.32 | 5.48 | 4.93 | 25.27 | 1.53 |
| Sample 17 | 0.54 | 0.73 | 0.37 | 2.62 | 21.82 | 14.15 | 40.23 | 22.92 |
| Sample 18 | 0.65 | 1.36 | 0.54 | 2.39 | 20.21 | 12.92 | 38.07 | 18.6 |
| Sample 19 | 0.79 | 2.2 | 0.77 | 2.18 | 18.91 | 11.75 | 36.6 | 9.64 |

Additional steviol glycosides are provided in Table 3.

TABLE 3
Summary of formula and R-groups of identified steviol glycosides

| | | | | | | |
|---|---|---|---|---|---|---|
| | | Trivial | Mol. | | | |
| # | Common name | formula | Wt. | R₁ | R₂ | Reference |

1. Steviol + Glucose (SvGn)

| | | | | | | |
|---|---|---|---|---|---|---|
| 1.1 | Steviolmonoside | SvG1 | 481 | H | Glcβ1- | Ohta et al. |
| | | | | | | (2010) |
| 1.2 | Steviolmonoside A | SvG1 | 481 | Glcβ1- | H | Gardena et |
| | | | | | | al. (2010) |
| 1.3 | Rubusoside | SvG2 | 643 | Glcβ1- | Glcβ1- | Ohta et al. |
| | | | | | | (2010) |
| 1.4 | Steviolbioside | SvG2 | 643 | H | Glcβ(1-2)Glcβ1- | Kohda et al. |
| | | | | | | (1976) |
| 1.5 | Stevioside | SvG3 | 805 | Glcβ1- | Glcβ(1-2)Glcβ1- | Bridel & |
| | | | | | | Lavielle |
| | | | | | | (1931) |
| 1.6 | Stevioside A | SvG3 | 805 | Glcβ(1-2)Glcβ1- | Glcβ1- | Wu et al. |
| | | | | | | (2012) |
| 1.7 | Rebaudioside B | SvG3 | 805 | H | Glcβ(1-2)[Glcβ(1- | Kohda et al. |
| | | | | | 3)]Glcβ1- | (1976) |
| 1.8 | Rebaudioside G | SvG3 | 805 | Glcβ1- | Glcβ(1-3)Glcβ1- | Ohta et al. |
| | | | | | | (2010) |
| 1.9 | Stevioside B | SvG3 | 805 | Glcβ(1-3)Glcβ1- | Glcβ1- | Chaturvedula |
| | | | | | | & Zamora |
| | | | | | | (2014) |
| 1.10 | Rebaudioside E | SvG4 | 967 | Glcβ(1-2)Glcβ1- | Glcβ(1-2)Glcβ1- | Sakamoto |
| | | | | | | et al. |
| | | | | | | (1977a) |
| 1.11 | Rebaudioside A | SvG4 | 967 | Glcβ1- | Glcβ(1-2)[Glcβ(1- | Kohda et al. |
| | | | | | 3)]Glcβ1- | (1976) |
| 1.12 | Rebaudioside A2 | SvG4 | 967 | Glcβ1- | Glcβ(1-6)Glcβ(1- | Chaturvedula |
| | | | | | 2)Glcβ1- | & Prakash |
| | | | | | | (2011d) |
| 1.13 | Rebaudioside D | SvG5 | 1 129 | Glcβ(1-2)Glcβ1- | Glcβ(1-2)[Glcβ(1- | Sakamoto |
| | | | | | 3)]Glcβ1- | et al. |
| | | | | | | (1977a) |
| 1.14 | Rebaudioside I | SvG5 | 1 129 | Glcβ(1-3)Glcβ1- | Glcβ(1-2)[Glcβ(1- | Ohta et al. |
| | | | | | 3)]Glcβ1- | (2010) |
| 1.15 | Rebaudioside L | SvG5 | 1 129 | Glcβ1- | Glcβ(1-6)Glcβ(1- | Ohta et al. |
| | | | | | 2)[Glcβ(1-3)]Glcβ1- | (2010) |
| 1.16 | Rebaudioside Q2 | SvG5 | 1 129 | Glcα(1-2)Glcα(1- | Glcβ(1-2)Glcβ1- | Chaturvedula |
| | | | | 4)Glcβ1- | | & Prakash |
| | | | | | | (2011c) |
| 1.17 | Rebaudioside Q | SvG5 | 1 129 | Glcβ1- | Glcα(1-4)Glcβ(1- | — |
| | | | | | 2)[Glcβ(1-3)]Glcβ1- | |
| 1.18 | Rebaudioside I2 | SvG5 | 1 129 | Glcβ1- | Glcα(1-3)Glcβ(1- | Chaturvedula |
| | | | | | 2)[Glcβ(1-3)]Glcβ1- | et al. |
| | | | | | | (2011c) |
| 1.19 | Rebaudioside Q3 | SvG5 | 1 129 | Glcβ1- | Glcα(1-4)Glcβ(1- | Chaturvedula |
| | | | | | 3)[Glcβ(1-2)]Glcβ1- | et al. |
| | | | | | | (2011c) |
| 1.20 | Rebaudioside I3 | SvG5 | 1 129 | Glcβ(1-2)[Glcβ(1- | Glcβ(1-2)Glcβ1- | Chaturvedula |
| | | | | 6)]Glcβ1- | | et al. |
| | | | | | | (2011c) |
| 1.21 | Rebaudioside M | SvG6 | 1 291 | Glcβ(1-2)[Glcβ (1- | Glcβ(1-2)[Glcβ(1- | Ohta et al. |
| | | | | 3)]Glcβ1- | 3)]Glcβ1- | (2010) |

2. Steviol + Rhamnose + Glucose (SvR1Gn)

| | | | | | | |
|---|---|---|---|---|---|---|
| 2.1 | Dulcoside A | SvR1G2 | 789 | Glcβ1- | Rhaα(1-2)Glcβ1- | Kobayashi |
| | | | | | | et al. (1977) |
| 2.2 | Dulcoside B | SvR1G2 | 789 | H | Rhaα(1-2)[Glcβ(1- | Ohta et al. |
| | | | | | 3)]Glcβ1- | (2010) |
| 2.3 | Rebaudioside C | SvR1G3 | 951 | Glcβ1- | Rhaα(1-2)[Glcβ(1- | Sakamoto |
| | | | | | 3)]Glcβ1- | et al. |
| | | | | | | (1977b) |
| 2.4 | Rebaudioside C2^{a} | SvR1G3 | 951 | Rhaα(1-2)Glcβ1- | Glcβ(1-2)Glcβ1- | Purkayastha |
| | | | | | | (2016) |
| 2.5 | Rebaudioside S | SvR1G3 | 951 | Rhaα(1-2)Glcβ1- | Glcα (1-2)Glcβ1- | Ibrahim et |
| | | | | | | al (2016) |
| 2.6 | Rebaudioside H | SvR1G4 | 1 112 | Glcβ1- | Glcβ(1-3)Rhaα(1- | Ohta et al. |
| | | | | | 2)[Glcβ(1-3)]Glcβ1- | (2010) |
| 2.7 | Rebaudioside K | SvR1G4 | 1 112 | Glcβ(1-2)Glcβ1- | Rhaα(1-2)[Glcβ(1- | Ohta et al. |
| | | | | | 3)]Glcβ1- | (2010) |
| 2.8 | Rebaudioside J | SvR1G4 | 1 112 | Rhaα(1-2)Glcβ1- | Glcβ(1-2)[Glcβ(1- | Ohta et al. |
| | | | | | 3)]Glcβ1- | (2010) |
| 2.9 | Rebaudioside N | SvR1G5 | 1 274 | Rhaα(1-2)[Glcβ(1- | Glcβ(1-2)[Glcβ(1- | Ohta et al. |
| | | | | 3)]Glcβ1- | 3)]Glcβ1- | (2010) |
| 2.10 | Rebaudioside O | SvR1G6 | 1 436 | Glcβ(1-3)Rhaα(1- | Glcβ(1-2)[Glcβ(1- | Ohta et al. |
| | | | | 2)[Glcβ(1-3)]Glcβ1- | 3)]Glcβ1- | (2010) |
| 2.11 | Rebaudioside O2^{a} | SvR1G6 | 1 436 | Glcβ(1-4*)Rhaα(1- | Glcβ(1-2)[Glcβ(1- | Purkayastha |
| | | | | 2)[Glcβ(1-3)]Glcβ1- | 3)]Glcβ1- | (2016) |
| 2.12 | Rebaudioside K2^{a} | SvR1G4 | 1 112 | Glcβ(1-6)Glcβ1- | Rhaα(1-2)[Glcβ(1- | Purkayastha |
| | | | | | 3)]Glcβ1- | (2016) |

3. Steviol + Xylose + Glucose (SvX1Gn)

| | | | | | | |
|---|---|---|---|---|---|---|
| 3.1 | Stevioside F | SvX1G2 | 775 | Glcβ1- | Xylβ(1-2)Glcβ1- | Chaturvedula |
| | | | | | | & Prakash |
| | | | | | | (2011b) |
| 3.2 | Rebaudioside F | SvX1G3 | 937 | Glcβ1- | Xylβ(1-2)[Glcβ(1- | Starratt et |
| | | | | | 3)]Glcβ1- | al. (2002) |
| 3.3 | Rebaudioside F2 | SvX1G3 | 937 | Glcβ1- | Glcβ(1-2)[Xylβ(1- | Chaturvedula |
| | | | | | 3)]Glcβ1- | & Prakash |
| | | | | | | (2011b) |
| 3.4 | Rebaudioside F3 | SvX1G3 | 937 | Xylβ(1-6)Glcβ1- | Glcβ(1-2)Glcβ1- | Chaturvedula |
| | | | | | | et al. |
| | | | | | | (2011d) |
| 3.5 | Rebaudioside R | SvX1G3 | 937 | Glcβ1- | Glcβ(1-2)[Glcβ(1- | Ibrahim et |
| | | | | | 3)] Xylβ1- | al (2016) |
| 3.6 | Rebaudioside U^{a} | SvX1G4 | 1 099 | Xylβ(1-2)Glcβ1- | Glcβ(1-2)[Glcβ(1- | Purkayastha |
| | | | | | 3)]Glcβ1- | (2016) |
| 3.7 | Rebaudioside U2^{a} | SvX1G4 | 1 099 | Xylβ(1-2*)[Glcβ(1- | Glcβ(1-2)Glcβ1- | Purkayastha |
| | | | | 3)]Glcβ1- | | (2016) |
| 3.8 | Rebaudioside V^{a} | SvX1G5 | 1 261 | Glcβ(1-2)[Glcβ(1- | Xylβ(1-2*)[Glcβ(1- | Purkayastha |
| | | | | 3)]Glcβ1- | 3)]Glcβ1- | (2016) |
| 3.9 | Rebaudioside V2^{a} | SvX1G5 | 1 261 | Xylβ (1-2)[Glcβ(1- | Glcβ(1-2)[Glcβ(1- | Prakash & |
| | | | | 3)]Glcβ1- | 3)]Glcβ1- | Chaturvedula |
| | | | | | | (2013) |

4. Steviol + Arabinose + Glucose (SvA1Gn)

| | | | | | | |
|---|---|---|---|---|---|---|
| 4.1 | Rebaudioside W^{a} | SvA1G4 | 1 098 | Glcβ(1-2)[Araβ(1- | Glcβ(1-2)Glcβ1- | Purkayastha |
| | | | | 3*)]Glcβ1 | | (2016) |
| 4.2 | Rebaudioside W2^{a} | SvA1G4 | 1 098 | Araβ(1-2*)Glcβ1 | Glcβ(1-2)[Glcβ(1- | Purkayastha |
| | | | | | 3)]Glcβ1- | (2016) |
| 4.3 | Rebaudioside W3^{a} | SvA1G4 | 1 098 | Araβ(1-6)Glcβ1- | Glcβ(1-2)[Glcβ(1- | Purkayastha |
| | | | | | 3)]Glcβ1- | (2016) |
| 4.4 | Rebaudioside Y^{a} | SvA1G5 | 1 260 | Glcβ(1-2)[Araβ(1- | Glcβ(1-2)[Glcβ(1- | Purkayastha |
| | | | | 3*)]Glcβ1 | 3)]Glcβ1- | (2016) |

5. Steviol + Fructose + Glucose (SvF1Gn)

| | | | | | | |
|---|---|---|---|---|---|---|
| 5.1 | Rebaudioside A3 | SvF1G3 | 967 | Glcβ1- | Glcβ(1-2)[Fruβ(1- | Chaturvedula |
| | | | | | 3)]Glcβ1- | et al. |
| | | | | | | (2011b) |

6. Steviol + galactose + Glucose (SvGa1Gn)

| | | | | | | |
|---|---|---|---|---|---|---|
| 6.1 | Rebaudioside T^{a} | SvGa1G4 | 1 128 | Galβ(1-2*)Glcβ1 | Glcβ(1-2)[Glcβ(1- | Purkayastha |
| | | | | | 3)]Glcβ1- | (2016)\hat{ } |

7. Steviol + de-oxy glucose + Glucose (SvdG1Gn)

| | | | | | | |
|---|---|---|---|---|---|---|
| 7.1 | Stevioside D | SvdG1G2 | 789 | Glcβ1- | 6-deoxyGlcβ(1- | Chaturvedula |
| | | | | | 2)Glcβ1- | & Prakash |
| | | | | | | (2011a) |
| 7.2 | Stevisoide E | SvdG1G3 | 951 | Glcβ1- | 6-deoxyGlcβ(1- | Chaturvedula |
| | | | | | 2)[Glcβ(1-3)]Glcβ1- | & Prakash |
| | | | | | | (2011a) |
| 7.3 | Stevioside E2 | SvdG1G3 | 951 | 6-deoxyGlcβ1- | Glcβ(1-2)[Glcβ(1- | Chaturvedula |
| | | | | | 3)]Glcβ1- | et al. |
| | | | | | | (2011e) |

### EXAMPLES

In the following Examples, Sample 7, having about 20% TCGA and about 15% TSG, Sample 16, having about 25% TCGA and less than 2% TSG, Sample 17, having about 40% TCGA and about 23% TSG, Sample 18 having about 38% TCGA and about 19% TSG, and Sample 19, having about 37% TCGA and about 10% TSG, as shown in Table 2 were used for the analyses. All test products containing either Sample 7 or Sample 16 or Sample 17 or Sample 18 or Sample 19 were analyzed against comparative products that did not contain any Stevia-derived natural flavor complex. The formulas for the comparative products that did not contain NFC were adjusted by increasing the amount of neutral ingredients in the formula which would not affect the sensory evaluation. The test and comparative products were evaluated across several attributes, including but not limited to sweetness intensity, bitter intensity, flavor (such as vanilla, cinnamon, strawberry, blueberry, chocolate), flavor notes (such as dairy, cream, whey, alcohol), sour intensity, mouthfeel, astringency, sweet aftertaste, bitter aftertaste, and overall liking.

### Example 1: Threshold Determination, FEMA Test 1

a. Water
a. 155 ppm of SAMPLE 7 in water provided sweetness perception significantly lower than 1.5% sugar solution. Therefore, we selected 155 ppm of SAMPLE 7 as the recognition threshold concentration.
b. 200 ppm of SAMPLE 17 in water provided sweetness perception significantly lower than 1.5% sugar solution. Therefore, we selected 200 ppm of SAMPLE 17 as the recognition threshold concentration.
c. 200 ppm of SAMPLE 18 in water provided sweetness perception significantly lower than 1.5% sugar solution. Therefore, we selected 200 ppm of SAMPLE 18 as the recognition threshold concentration.
d. 200 ppm of SAMPLE 19 in water provided sweetness perception significantly lower than 1.5% sugar solution. Therefore, we selected 200 ppm of SAMPLE 19 as the recognition threshold concentration.

b. Baked Goods
a. 600 ppm of SAMPLE 7 in Blueberry Cinnamon Muffin provided sweetness perception significantly lower than that of 4.0% sugar solution. Therefore, we selected 600 ppm of SAMPLE 7 as the recognition threshold concentration.

c. Breakfast Cereal
a. 1000 ppm of SAMPLE 7 in strawberry breakfast cereal provided sweetness perception significantly lower than that of 3.0% sugar solution. Therefore, we selected 1000 ppm of SAMPLE 7 as the recognition threshold concentration.

d. Milk/Dairy Products
a. 165 ppm of SAMPLE 7 in chocolate protein shake with 2% milk provided sweetness perception significantly lower than 1.5% sugar solution. Therefore, we selected 165 ppm of SAMPLE 7 as the recognition threshold concentration.

e. Imitation Dairy Products
a. 165 ppm of SAMPLE 7 in chocolate protein shake with almond milk provided sweetness perception significantly lower than 1.5% sugar solution. Therefore, we selected 165 ppm of SAMPLE 7 as the recognition threshold concentration.

### Example 2: Descriptive Analysis, FEMA Test 2 of Sample 7

a. Non-Dairy/Imitation Dairy Products:
a. Vanilla Cinnamon Oat milk (SAMPLE 7 usage level 125 ppm). The results indicate that test sample containing SAMPLE 7 to be significantly higher vanilla flavor, and oat flavor than the control (at 95% confidence, and 90% confidence respectively).
b. The test sample had significantly lower bitter intensity and directionally lower aftertaste (sweet & bitter) at 90% confidence and 80% confidence, respectively.

b. Milk/Dairy Products:
a. Chocolate protein shake (SAMPLE 7 usage level 125 ppm). Results indicate the addition of the modifier significantly lower whey protein note, and bitter aftertaste than the control (at 95% confidence).
b. Results indicated that test sample has higher cocoa flavor, and cream notes (at 95% confidence).

c. Non-Alcoholic Beverage:
a. Brewed tea with lemon (SAMPLE 7 using level 125 ppm). Results indicate test sample had lower bitterness, and lower aftertaste (sweet and bitter) than the control (at 95% confidence).
b. Results indicated the test sample had higher brewed team flavor and overall liking (at 95% confidence).

d. Alcoholic Beverage:
a. Vodka with Lemonade (SAMPLE 7 usage level 125 ppm). Results indicate test sample had significantly higher lemon flavor, alcohol note than the control (at 95% confidence).
b. Test samples had lower sweet aftertaste and bitter aftertaste (at 90% confidence, and 95% confidence respectively)

e. Baked Goods:
a. Blueberry Cinnamon Muffins (SAMPLE 7 usage level 500 ppm). Results indicate test samples had significantly higher blueberry flavor, cinnamon flavor, and brown note than control (at 95% confidence).
b. Test sample had lower sweet aftertaste and bitter aftertaste (at 95% confidence, and 90% confidence respectively).

f. Jams & Jellies:
a. Strawberry Jam (SAMPLE 7 usage level 125 ppm). Results indicate test sample had significantly higher strawberry flavor, fresh strawberry flavor than the control (at 95% confidence).
   i. Test sample had significantly lower aftertaste (sweet and bitter) (at 90% confidence).

g. Breakfast Cereals:
i. Strawberry Cereal (SAMPLE 7 usage level 1000 ppm). Results indicate test sample had significantly higher strawberry flavor (at 90% confidence).
ii. Test sample had significantly lower aftertaste (sweet and bitter) (at 90% confidence).

h. Fats & Oils:
i. Soy Ginger Dressing (SAMPLE 7 usage level 125 ppm). Results indicate test sample had directionally lower bitterness (at 80% confidence).
ii. Sesame Ginger Dressing (SAMPLE 7 usage level 155 ppm). Results indicate test sample had directionally lower astringency, total flavor intensity, vinegar, sesame, garlic flavor, and higher in shape/body (mouthfeel) and sweet taste (at 95% confidence).
iii. Sesame Ginger Dressing (SAMPLE 19 usage level 155 ppm). Results indicate test sample had directionally lower astringency, total flavor intensity, vinegar, sesame, garlic flavor, and higher in shape/body (mouthfeel) and sweet taste (at 95% confidence).

### Example 3: Descriptive Analysis, FEMA Test 2 of Sample 16

a. Non-Dairy/Imitation Dairy Products:
i. Vanilla Cinnamon Oat milk (SAMPLE 16 usage level 125 ppm). The results indicate that test sample containing SAMPLE 16 to be significantly higher vanilla flavor, and oat flavor than the control (at 95% confidence, and 90% confidence respectively).
iv. The test sample had significantly lower bitter intensity and directionally lower aftertaste (sweet & bitter) at 90% confidence and 80% confidence, respectively.

b. Non-Alcoholic Beverage:
i. Brewed tea with lemon (SAMPLE 16 using level 125 ppm). Results indicate test sample had lower bitterness, astringency, and lower bitter aftertaste than the control (at 95% confidence).
ii. Results indicated the test sample had higher brewed tea flavor and lower sweet aftertaste (at 80% confidence).

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition. If there is any conflict in the usages of a word or term in this specification and one or more patent or other documents that may be incorporated herein by reference, the definitions that are consistent with this specification should be adopted.

What is claimed is:

1. A method for modifying a flavor of a consumable product comprising adding to the consumable product a stevia extract flavor with modifying properties (FMP) comprising chlorogenic acid in an amount from about 10 wt % to about 50 wt % and total steviol glycosides in an amount from greater than 0 wt % to about 25 wt %, wherein the stevia extract FMP modifies the flavor and sweetness profile of the consumable product without imparting detectable sweetness to the consumable product.

2. The method of claim 1, wherein the chlorogenic acid comprises at least one chlorogenic acid molecule selected from the group consisting of neo-chlorogenic acid (neo-CGA; 5-O-caffeoylquinic acid or 5-CQA), crypto-chlorogenic acid (crypto-CGA; 4-O-caffeoylquinic acid or 4-CQA), n-chlorogenic acid (n-CGA; 3-O-caffeoylquinic acid or 3-CQA), iso-chlorogenic acid A (iso-CGA A; 3,5-dicaffeoylquinic acid) iso-chlorogenic acid B (iso-CGA B; 3,4-dicaffeoylquinic acid), and iso-chlorogenic acid C (iso-CGA C; 4,5-dicaffeoylquinic acid).

3. The method of claim 1, wherein the total steviol glycosides comprise at least one steviol glycoside molecule selected from the group consisting of Rebaudioside A, Rebaudioside B, Rebaudioside C, Rebaudioside D, Rebaudioside E, Rebaudioside F, Rebaudioside AM, Rebaudioside M, Rebaudioside N, Rebaudioside O, Stevioside, Steviolbioside, Dulcoside A, Rubusoside, and minor steviol glycosides.

4. The method of claim 1, wherein the stevia extract FMP further comprises at least one molecule selected from the group consisting of Tricaffeoylquinic acids; Rutin; Feruloyl-caffeoylquinc acids; Caffeoyl-feruloylquinic acids; Quercetin-glycoside; Galuteolin; Quercitrin; Roseoside; Feruloylquinic acids; Coumaroylquinic acids; Caffeoylshikimic acids; Caffeoylshikimic acids; 5-Caffeoylshikimic acid; Catechin; Luteolin; Trans-ferulic acid; and combinations thereof.

5. The method of claim 1, wherein the stevia extract FMP further comprises at least one molecule selected from the group consisting of flavonoids, retinoids, pigments, polysaccharides, oligosaccharides, disaccharides, monosaccharides, volatile oil components, sterols, terpenoids, sesquiterpenoids, diterpenes, triterpenes, coumarins, fatty acids and their derivatives, amino acids and their derivatives, dipeptides, oligopeptides, polypeptides, proteins, austroinulin, quercetin, sterebins, spathulenol, decanoic acid, 8,11,14-ecosatrienoic acid, 2-methyloctadecane, pentacosane, octacosane, stigmasterol, bsitosterol, a- and b-amyrine, Iupeol, b-amyrin acetate, pentacyclic triterpene and glycosides thereof, and combinations thereof.

6. The method of claim 1, wherein the stevia extract FMP is added to the consumable product in an amount below a stevia extract FMP sweetness detection threshold level.

7. The method of claim 6, wherein the stevia extract FMP is added to the consumable product in an amount ranging from 25 ppm to 1500 ppm.

8. The method of claim 1, wherein the flavor of the consumable product is modified by reducing bitterness or astringency or sweet aftertaste or bitter aftertaste perception of the consumable product as compared to a control product that does not contain the stevia extract FMP.

9. The method of claim 1, wherein the flavor of the consumable product is modified by enhancing one or more flavor notes as compared to a control product that does not contain the stevia extract FMP.

10. The method of claim 1, wherein the flavor of the consumable product is modified by suppressing one or more flavor notes as compared to a control product that does not contain the stevia extract FMP.
